(19) United States
(12) Patent Application Publication
(10) Pub. No.: US 2023/0172456 A1
(43) Pub. Date: Jun. 8, 2023

(54) Installation State Determination Method, and Installation State Determination System

(71) Applicant: Nippon Telegraph and Telephone Corporation, Tokyo (JP)

(72) Inventors: Daichi Matsunaga, Tokyo (JP); Yujiro Tanaka, Tokyo (JP); Michiko Seyama, Tokyo (JP)

(21) Appl. No.: 17/925,747

(22) PCT Filed: May 28, 2020
(86) PCT No.: PCT/JP2020/021092

§ 371 Date: Nov. 16, 2022

(51) Int. Cl. A61B 5/01; A61B 5/00

(52) U.S. Cl. CPC A61B 5/01; A61B 5/6843; A61B 5/7246

(57) ABSTRACT

An installation state determination method of the present disclosure includes measuring a temperature and a heat flux of a surface of a living body using a sensor installed at a predetermined site of the living body, calculating a thermal resistance value of the living body based on the measured temperature and heat flux of the surface of the living body, comparing the calculated thermal resistance value of the living body with a reference thermal resistance value of the predetermined site of the living body, and determining an installation state of the sensor at the predetermined site of the living body based on a result of the comparison.

### CROSS-REFERENCE TO RELATED APPLICATIONS

This application is a national phase entry of PCT Application No. PCT/JP2020/021092, filed on May 28, 2020, which application is hereby incorporated herein by reference.

### TECHNICAL FIELD

The present disclosure relates to an installation state determination method of a sensor in a temperature measurement technique for measuring a core body temperature of a living body.

### BACKGROUND

A technique for acquiring biological information such as circadian rhythms by continuously measuring core body temperatures that are temperatures of the core part of a living body has been proposed in the related art. For example, NPL1 and NPL 2 relate to a non-invasive technique to estimate a core body temperature using a body surface temperature measured with a temperature sensor on the assumption of a thermal equivalent circuit that is created by replacing the course of heat transmission in a living body with an electrical circuit.

FIG. 10 is a thermal equivalent circuit of a temperature measuring device for estimating a core body temperature of a living body using a dual heat flux method. Two probes 310 and 320 are disposed on a surface of a living body 400. The probes 310 and 320 included in a temperature measuring device 300 include thermal insulation members (thermal resistors R1 and R2) having different thermal resistances from each other. The probe 310 measures a body surface temperature T1 and a body surface heat flux H1 via the thermal insulation member R1. The probe 320 measures a body surface temperature T2 and a body surface heat flux H2 via the thermal insulation member R2.

A core body temperature Tc is expressed by Equation (2). Here, Rb denotes a thermal resistance of the living body 400, which is an unknown value.

$$\text{Tc = T1} + \text{Rb} \cdot \text{H1}$$

$$\text{Tc = T2} + \text{Rb} \cdot \text{H2}$$

The core body temperature Tc is expressed by Equation (3) by using the above Equation (2). It is possible to estimate the core body temperature Tc using the following Equation (3).

$$\text{Tc =} \left(\text{T2} \cdot \text{H1} - \text{T1} \cdot \text{H2}\right) / \left(\text{H1} - \text{H2}\right)$$

Here, because the living body 400 actually includes continuous tissues and is combined with adjacent tissues, leakage of heat fluxes (HL1 and HL2) may take place as illustrated in FIG. 10. The leakage of the heat fluxes (HL1 and HL2) takes place inside the living body 400, and thus cannot be measured. For this reason, a technique to estimate the core body temperature Tc more accurately by performing calibration in estimation of the core body temperature Tc has been proposed.

The core body temperature Tc obtained by taking leakages of the heat fluxes (HL1 and HL2) from the probes 310 and 320 into account can be expressed by Equation (4).

$$\text{Tc = T1} + \text{Rb} \cdot \left(\text{H1+HL1}\right)$$

$$\text{Tc = T2} + \text{Rb} \cdot \left(\text{H2+HL2}\right)$$

The core body temperature Tc can be expressed by Equation (5) using the above-described Equation (4).

$$\text{Tc =} \left(\text{K} \cdot \text{T2} \cdot \text{H1} - \text{T1} \cdot \text{H2}\right) / \left(\text{K} \cdot \text{H1} - \text{H2}\right)$$

Here, K is a proportion of the leakages of the heat fluxes from the two probes 310 and 320 and is expressed by Equation (6).

$$\text{K} = \left(\left(\text{H1} \text{+} \text{HL1}\right) / \text{H1}\right) / \left(\left(\text{H2} \text{+} \text{HL2}\right) / \text{H2}\right)$$

Here, because the leakages of the heat fluxes (HL1 and HL2) are spread inside the living body and cannot be measured, K is initially calibrated using a reference core body temperature Tc(o) at a time t(o) as expressed by Equation (7). The reference core body temperature Tc(0) is a known value obtained using another method.

$$\text{K} \left(\text{o}\right) = \left(\left(\text{Tc} \left(\text{o}\right) \text{-} \text{T1} \left(\text{o}\right)\right) / \text{H1} \left(\text{o}\right)\right) / \left(\left(\text{Tc} \left(\text{o}\right) \text{-} \text{T2} \left(\text{o}\right)\right) / \text{H2} \left(\text{o}\right)\right)$$

According to the techniques of NPL 1 and NPL 2, the temperature measuring device 300 includes a thermal conduction member 330 that covers the peripheries of the probes 310 and 320 being in contact with the surface of the living body 400 to measure temperatures. A bridge circuit of the thermal equivalent circuit of this case is illustrated in FIG. 11. When the thermal conduction member 330 is provided, a change in the thermal resistance Ra of the outside air does not affect the proportion K of the leakage of the heat fluxes, and thus even when a convection state of the outside air changes and the thermal resistance Ra changes, it is possible to estimate the core body temperature Tc that is not dependent on the thermal resistance Ra.

### CITATION LIST

### Non Patent Literature

NPL 1: Matsunaga et al. (2019) “Study on Non-Invasive Core Body Temperature Estimation Method for Convection Change in Outside Air,” Communication Society Conference of Institute of Electronics, Information and Communication Engineers, September 10 to 13, 2019.

NPL 2: Matsunaga et al. (2020), “Study for Miniaturization of Non-Invasive Core Body Temperature Sensor Considering Convection Change,” General Conference of Institute of Electronics, Information and Communication Engineers, March 17 to 20, 2020.

### SUMMARY

### Technical Problem

However, in the techniques of NPL 1 and NPL 2, an air layer formed between the temperature measuring device 300 and the living body 400 when the temperature measuring device 300 is installed on the living body 400 makes a difference from the designed thermal equivalent circuit, which leads to a problem that accuracy in the measurement of a core body temperature deteriorates when the convection state of the outside air changes.

The present disclosure has been made to solve the problems described above, and aims to provide an installation state determination method capable of determining an installation state of a sensor of a temperature measuring device in a living body and notifying a user of incorrect installation of the sensor.

### Means for Solving the Problem

To solve the above-described problems, an installation state determination method according to the present disclosure includes measuring a temperature and a heat flux of a surface of a living body using a sensor installed at a predetermined site of the living body, calculating a thermal resistance value of the living body based on the measured temperature and heat flux of the surface of the living body, comparing the calculated thermal resistance value of the living body with a reference thermal resistance value of the predetermined site of the living body, and determining an installation state of the sensor at the predetermined site of the living body based on a result of the comparison.

In addition, to solve the above-described problems, an installation state determination system according to the present disclosure includes a sensor that is installed at a predetermined site of a living body and measures a temperature and a heat flux of a surface of the living body, and a computation device that calculates a thermal resistance value of the living body based on the measured temperature and heat flux of the surface of the living body and compares the calculated thermal resistance value of the living body with a reference thermal resistance value of the predetermined site of the living body, in which the computation device determines an installation state of the sensor at the predetermined site of the living body based on a result of the comparison.

### Effects of Embodiments of the Invention

According to the present disclosure, it is possible to provide an installation state determination method capable of determining an installation state of a sensor of a temperature measuring device in a living body and notifying a user of incorrect installation of the sensor.

### BRIEF DESCRIPTION OF THE DRAWINGS

FIG. 1 is a diagram illustrating a thermal equivalent circuit of a temperature measuring device according to an embodiment of the present disclosure.

FIG. 2 is a diagram illustrating a bridge circuit of the thermal equivalent circuit of the temperature measuring device according to the present embodiment.

FIG. 3 is a diagram illustrating the thermal equivalent circuit of the temperature measuring device when there is an air layer.

FIG. 4 is a diagram illustrating a bridge circuit of the thermal equivalent circuit of the temperature measuring device when there is an air layer.

FIG. 5 is a diagram for explaining a temperature measurement error when there is an air layer.

FIG. 6 is a diagram for explaining a relationship between a thermal resistance of a living body and a thickness thereof.

FIG. 7 is a diagram illustrating a configuration example of the temperature measuring device according to the embodiment of the present disclosure.

FIG. 8 is a block diagram illustrating a configuration example of an installation state determination system according to the embodiment of the present disclosure.

FIG. 9 is a flowchart of an operation of an installation state determination method according to the embodiment of the present disclosure.

FIG. 10 is a diagram illustrating a thermal equivalent circuit of a temperature measuring device of the related art.

FIG. 11 is a diagram illustrating a bridge circuit of the thermal equivalent circuit of the temperature measuring device of the related art.

### DETAILED DESCRIPTION OF ILLUSTRATIVE EMBODIMENTS

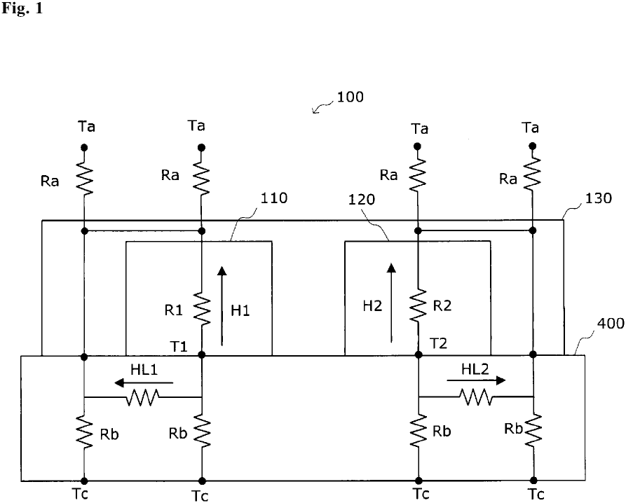

Hereinafter, a preferred embodiment of the present disclosure will be described in detail with reference to FIGS. 1 to 9.

### Temperature Measurement of Temperature Measuring Device

First, temperature measurement of a temperature measuring device used in an installation state determination method and an installation state determination system of the present disclosure will be described.

The temperature measuring device used in the installation state determination method and the installation state determination system of the present disclosure includes a first probe that measures a physical quantity relating to a temperature of a substance based on a first reference and a second probe that measures a physical quantity related to a temperature of the substance based on a second reference, and a thermal conduction member that covers the first probe and the second probe and transports heat from the substance. When the temperature measuring device is applied to a system for measuring a core body temperature of a living body, the first and second probes have thermal resistances (the first reference and the second reference) and measure body surface temperatures and body surface heat fluxes of the epidermis of the living body 400.

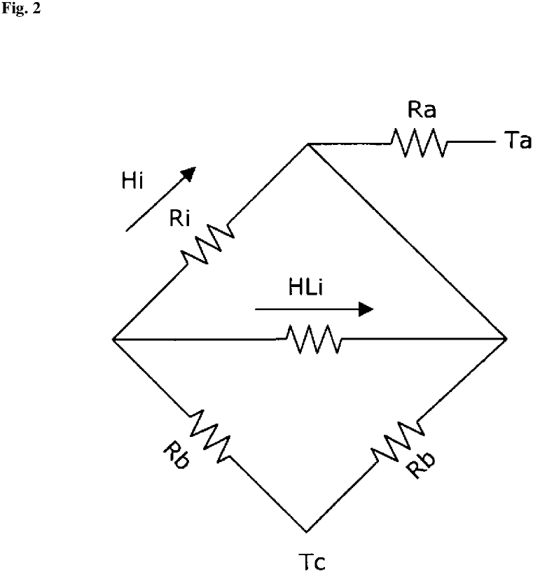

FIGS. 1 and 2 illustrate a thermal equivalent circuit of a temperature measuring device 100 used in the installation state determination method and the installation state determination system of the present disclosure. The temperature measuring device 100 in FIG. 1 includes two probes (a first probe and a second probe) 110 and 120 disposed to come in contact with the epidermis of the living body 400. The probes 110 and 120 differ from each other in thermal resistance (the first reference and the second reference) and measure body surface temperatures and surface heat fluxes of the epidermis of the living body 400.

In the thermal equivalent circuit including the living body 400, the probe 120, the thermal conduction member 130, and outside air, Ta represents an outside air temperature, Ra represents a thermal resistance against outside air, R1 represents a known value of a thermal resistance of the probe 120, H1 represents a heat flux in the thermal resistance R1, Rb represents a thermal resistance of the living body 400, and HL1 represents leakage of the heat flux taking place inside the living body 400.

The thermal conduction member 130 that does not come in contact with the epidermis of the living body 400 is provided to cover the outer peripheral surfaces of the two probes 110 and 120. For this reason, even when a convection state of the outside air changes and the thermal resistance Ra changes, it is possible to estimate the core body temperature Tc that is not dependent on the thermal resistance Ra.

The thermal conduction member 130 is made of a material having a higher thermal conductivity than outside air, and transports heat transmitted from the surface of the living body 400 to make the temperature around the probes 110 and 120 equal to the temperature of the surface of the living body 400.

The probe 120 illustrated in FIG. 1 is joined to surrounding thermal resistances to form the bridge circuit illustrated in FIG. 2. In the bridge circuit illustrated in FIG. 2, the thermal conduction member 130 is provided, so that the thermal resistance Ra against the outside air is connected on the outside of the bridge circuit. Thus, even when the thermal resistance Ra against the outside air changes, the proportion of leakages of the heat fluxes (HL1 and HL2) does not change.

The temperature measuring device 100 according to the present embodiment includes the thermal conduction member 130 that covers the peripheries of the probes 110 and 120 being in contact with the surface of the living body 400 to measure temperatures and thus can estimate the core body temperature Tc that is not dependent on the thermal resistance Ra even when a convection state of the outside air changes and thus the thermal resistance Ra changes.

### Thermal Equivalent Circuit When There is Air Layer

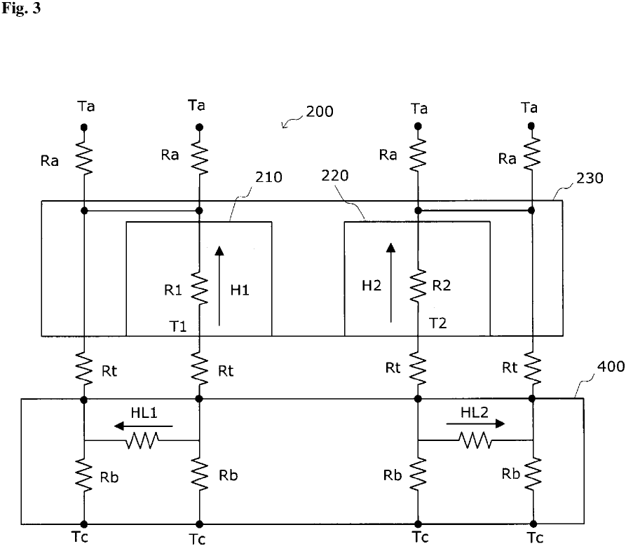

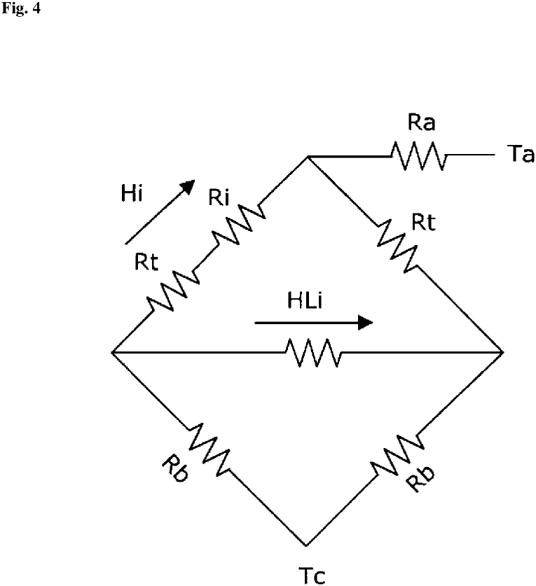

Next, a thermal equivalent circuit when there is an air layer formed between a sensor of a temperature measuring device 200 according to the present embodiment and the living body 400 will be described. FIGS. 3 and 4 illustrate a thermal equivalent circuit and a bridge circuit of the temperature measuring device 200 when there is an air layer formed between the temperature measuring device and the living body 400.

In the thermal equivalent circuit of FIG. 3, the air layer is formed between the sensor of the temperature measuring device 200 and the living body 400, and thermal resistances Rt of the air layer are disposed. Probes 210 and 220 are joined to the surrounding thermal resistances to form the bridge circuit illustrated in FIG. 4.

In the bridge circuit of FIG. 4, when a convection state of the outside air is changed, the thermal resistances Rt formed in the air layer change, which changes the balance between the heat fluxes in the probes and leakage of the heat fluxes in the living body. In other words, H1, H2, HL1, and HL2 change in Equation (5) described above, and consequently, accuracy in measurement of a core body temperature may deteriorate when a convection state of the outside air changes.

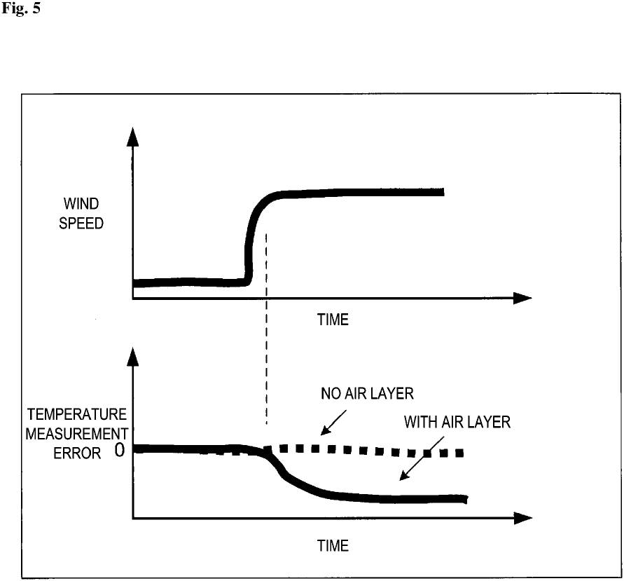

FIG. 5 is a diagram for explaining a temperature measurement error when there is an air layer. When there is an air layer between the sensor of the temperature measuring device 200 and the living body 400, the thermal resistances Rt of the air layer change due to the influence of the air velocity of the outside air as shown in FIG. 5, the balance between the heat fluxes in the probes and leakages of the heat fluxes in the living body changes, and thus accuracy in measurement of a core body temperature deteriorates.

Thus, the present embodiment is configured such that a thermal resistance value of the living body at the time of initial calibration, which is calculated based on an actually measured body surface temperature and body surface heat flux, is compared with a reference thermal resistance value at a predetermined thickness of the living body to determine whether an air layer is present between the temperature measuring device 200 and the living body 400.

Specifically, the thermal resistance value (Tc(0) - T1(0))/H1(0) in Equation (7) used in the initial calibration is compared to the reference thermal resistance value of the predetermined thickness of the living body, and whether an air layer is formed is determined based on the comparison result.

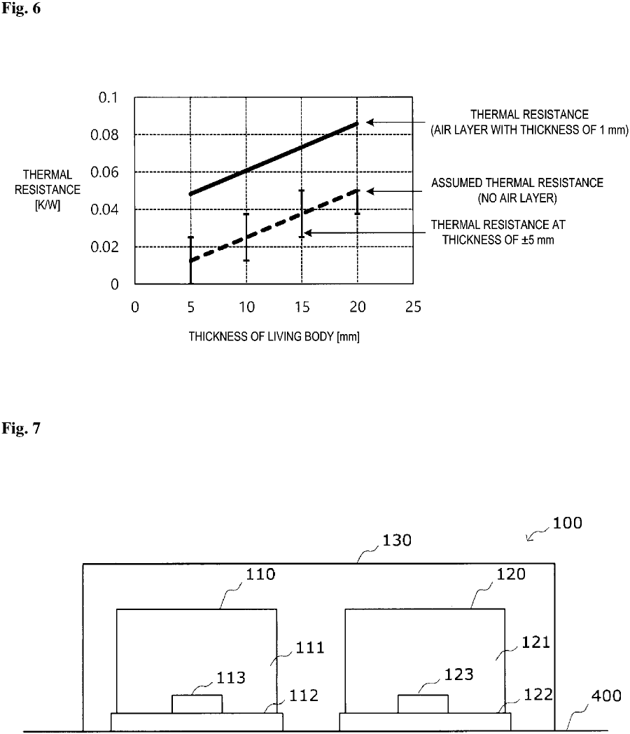

FIG. 6 illustrates an example of the relationship between a thermal resistance and a thickness of the living body in cases in which the air layer has a thickness of 1 mm and no air layer is present. If there is no air layer and an approximate thickness of a measurement site of the living body is known, a value of the assumed thermal resistance at the site can be understood in advance. Further, the thermal conductivity of the air (0.028 W/(m·K)) is 14 times lower than the thermal conductivity of the living body (0.4 W/(m·K)).

Using this feature, the value of the assumed thermal resistance of the measurement site of the living body having a known approximate thickness and a threshold of the thermal resistance are set, whether the difference between the value of the thermal resistance of the living body at the time of initial calibration and the value of the assumed thermal resistance exceeds the predetermined threshold is determined, and thus it is possible to determine whether there is an air layer on the sensor attaching surface.

For example, when the measurement site is the forehead, the thickness of the forehead is assumed to be 10 ± 5 mm, and thus the value of the thermal resistance of an error of this thickness is set as a threshold. Then, it is possible to determine whether there is an air layer on the probe attaching surface based on whether the difference between the value of the thermal resistance of the living body at the time of initial calibration and the value of the assumed thermal resistance exceeds the threshold value.

In the example shown in FIG. 6, in the case in which a thermal resistance 0.04 (K/W) is set as a threshold value for the living body with a thickness of 10 mm and the value of the thermal resistance of the living body at the time of initial calibration exceeds the thermal resistance 0.04 (K/W), it is determined that there is an air layer. Because values of the assumed thickness of the living body and the threshold differ according to a site of the living body on which a sensor is installed, such values are only required to be set in advance according to a sensor installation site.

Because the present embodiment is configured such that whether there is an air layer between the probes and the living body is determined using the value of the thermal resistance of the living body calibrated when the probes are attached, it is possible to notify a user of whether the probes are correctly attached and to prompt the user to attach the probes again.

Further, because a core temperature of the living body is estimated based on body surface temperatures and body surface heat fluxes actually measured using the two probes in the present embodiment, there may be an error in measurement of the core temperature if an air layer is formed between the sensor of any one of the two probes and the living body. For this reason, if it is determined that there is an air layer between at least one of the two probes and the living body, the probe needs to be attached again.

### Configuration of Temperature Measuring Device

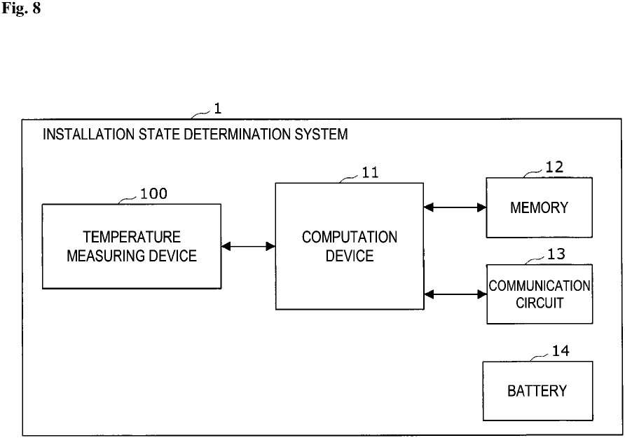

A configuration example of the temperature measuring device used in the installation state determination method and the installation state determination system of the present disclosure will be described using FIG. 7. The temperature measuring device 100 includes the two probes 110 and 120, and the thermal conduction member 130 that is made of aluminum and covers the peripheries of the probes 110 and 120. The probes 110 and 120 include thermal insulation members (a first thermal resistor and a second thermal resistor) 111 and 121, heat flux sensors (a first heat flux measurement unit and a second heat flux measurement unit) 112 and 122, and temperature sensors (a first temperature measurement unit and a second temperature measurement unit) 113 and 123, respectively.

The thermal insulation members 111 and 121 constitute thermal resistors having different thermal resistance values from each other. The thermal insulation members 111 and 121 may have the same rectangular parallelepiped shape formed of, for example, different materials from each other. Alternatively, the thermal insulation members 111 and 121 may be formed of a thermal insulation material having different thicknesses and materials to have different thermal resistance values from each other.

The heat flux sensors 112 and 122 are devices that measure movement of heat per unit time and unit area. The heat flux sensors 112 and 122 are provided at ends of the thermal insulation members 111 and 121 to face the epidermis of the living body 400.

The temperature sensors 113 and 123 measure temperatures of the surface of the living body 400. The temperature sensors 113 and 123 can be configured as thermistors, thermocouples, temperature measuring resistors, or the like.

The outer peripheral surfaces of the probes 110 and 120 are covered with the thermal conduction member 130 formed of a material having a high thermal conductivity like a metal such as aluminum or copper or a graphene sheet, or the like. The outside air does not come in direct contact with the probes 110 and 120, but comes in contact with the thermal conduction member 130.

The thermal conduction member 130 has a function of making the temperature of the body surface of the living body 400 in contact with the probes 110 and 120 equal to the temperature of the surface of the living body 400 not in contact with the probes 110 and 120 and the temperature around the surface, that is, a function of forming an isothermal region. As such an isothermal region is formed, a desired thermal equivalent circuit as illustrated in FIG. 1 is established.

The thickness of the thermal conduction member 130 around the probes 110 and 120 may be the optimal thickness to form an isothermal region in consideration of the thermal resistance Ra to the outside air, the thermal resistance Rb of the living body 400, and the like. More specifically, the thickness can be determined considering the surface area of the probes 110 and 120, the site, blood flow, and the like of the living body 400.

Further, in FIG. 7, although the configuration example in which the probes 110 and 120 have the temperature sensors 113 and 123 and the heat flux sensors 112 and 122, respectively, has been described, the temperature measuring device may have other configuration as long as it enables each of the probes to measure a temperature and heat flux of a living body. For example, temperature sensors may be installed on the upper and lower parts of the probes to calculate the fluxes using the measurement results of the temperature sensors.

### Configuration of Installation State Determination System

FIG. 8 is a block diagram illustrating a configuration example of an installation state determination system 1 according to the present disclosure. The installation state determination system 1 of the present disclosure measures temperatures and heat fluxes of a living body using the temperature measuring device 100 with the above-described configuration.

The installation state determination system 1 includes the temperature measuring device 100, a computation device 11, a memory 12, a communication circuit 13 that functions as an I/F circuit with respect to the outside, and a battery 14 that supplies power to the computation device 11, the communication circuit 13, and the like.

The computation device 11 estimates a core body temperature of the living body 400 based on heat fluxes measured by the heat flux sensors 112 and 122 and the body surface temperatures measured by the temperature sensors 113 and 123. More specifically, the computation device 11 estimates a core body temperature Tc using the above-described Equations (5) to (7). In estimating the core body temperature Tc, initial calibration is performed using Equation (7).

Furthermore, the computation device 11 compares the value of the thermal resistance of the living body 400 at the time of initial calibration with the reference thermal resistance value at a predetermined thickness of the living body. Specifically, the computation device 11 compares the value of the thermal resistance of the living body 400 at the time of initial calibration in the above-described Equation (7) with the reference thermal resistance value at the predetermined thickness and outputs the comparison result.

The memory 12 stores information regarding the pre-constructed estimation models of the core body temperature Tc (Equations (5) to (7)). Furthermore, the memory 12 stores in advance the thermal resistance value of each of the probes 110 and 120, the reference core body temperature of the living body used at the time of initial calibration, and the reference thermal resistance value at the predetermined thickness.

The memory 12 can be implemented by a predetermined storage area in a rewritable non-volatile storage device (e.g., a flash memory, etc.) provided in the installation state determination system 1.

The communication circuit 13 outputs, to the outside, the comparison result of the core body temperature Tc of the living body 400 generated by the computation device 11 and the value of the thermal resistance. As such a communication circuit 13, an output circuit to which a USB or other cable can be connected can be used when data or the like is output by wire. A wireless communication circuit using Bluetooth (trade name) or the like may be used.

Further, although not illustrated, the installation state determination system 1 includes a sheet-like base material that functions as a foundation for placing the temperature measuring device 100, the computation device 11, the memory 12, the communication circuit 13, and the battery 14 thereon, and wires that electrically connect these devices.

The installation state determination system 1 is achieved by a computer. Specifically, the computation device 11 is achieved, for example, by a processor such as a CPU or a DSP executing various types of data processing in a program stored in a storage device such as a ROM, a RAM, and a flash memory including the memory 12 provided in the installation state determination system 1. The program for causing a computer to function as the installation state determination system 1 can be recorded on a recording medium or provided via a network.

### Operation of Installation State Determination Method

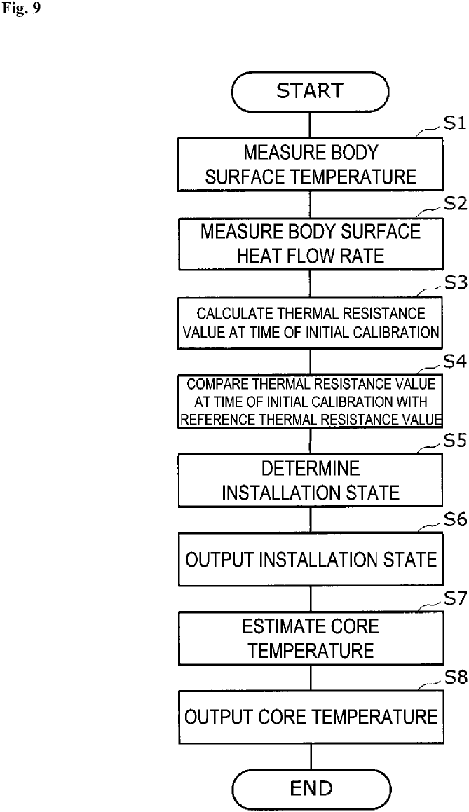

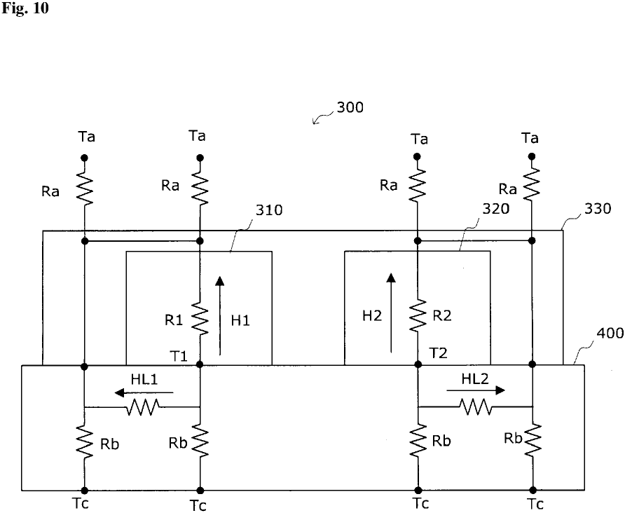

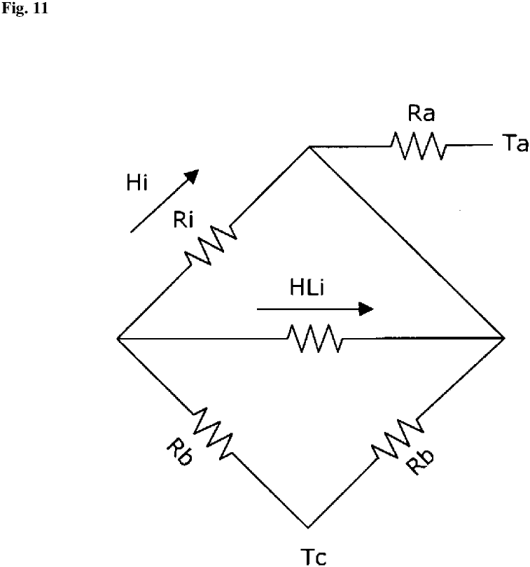

An installation state determination method performed by the installation state determination system 1 of the present disclosure will be described below with reference to the flowchart of FIG. 9. In the installation state determination method according to the present embodiment, the temperature measuring device 100 is installed to be in contact with the epidermis of the living body 400 in advance to perform the following processing.

First, each of the temperature sensors 113 and 123 measures the temperature of the surface of the living body 400 (step S1). The measured temperatures are stored in the memory 12.

Next, each of the heat flux sensors 112 and 122 measures the heat flux of the surface of the living body 400 (step S2). The values of the measured heat fluxes are stored in the memory 12.

Then, the computation device 11 reads the model for estimating the core body temperature from the memory 12 (Equations (5) to (7)) and then performs initial calibration based on the measured temperatures and heat fluxes of the surface of the living body and the reference core body temperature of the living body at the time of starting the measurement (step S3).

In the present embodiment, the value of the thermal resistance of the living body 400 used at the time of the initial calibration is compared with the reference thermal resistance value at a predetermined thickness (step S4), and the installation state of the sensor at the measurement site of the living body 400 is determined based on the result of the comparison (step S5).

The comparison result (determination result) of the values of the thermal resistances is output from the communication circuit 13 (step S6). For example, the comparison result (determination result) of the values of the thermal resistances can be transmitted to an external terminal via a communication network.

The external terminal notifies the user of whether the sensor of the living body temperature measuring device is correctly attached to the living body, using the comparison result of the values of the thermal resistances. As a result, the user can attach the sensor to the living body again when the sensor is not correctly attached to the living body.

Then, the computation device 11 reads the model for estimating the core body temperature from the memory 12 (Equations (5) to (7)) and inputs the measured temperatures and heat fluxes of the surface of the living body into the estimation model to estimate the core body temperature (step S7). The estimated core body temperature is output from the communication circuit 13 to the external device so that time-series data of the core body temperature is collected (step S8).

Further, although the comparison result (determination result) of the values of the thermal resistances is output from the communication circuit 13 in the above-described embodiment, it may be configured such that whether the sensor of the temperature measuring device is correctly attached to the living body is displayed based on the comparison result of the values of the thermal resistances in the installation state determination system 1. For example, the installation state determination system 1 may be configured to include a display device such as an LED to display the comparison result (determination result) on the display device.

Further, although the computation device 11 performs the comparison of the values of the thermal resistances of the living body used in the initial calibration with the reference thermal resistance value at the predetermined thickness in the above-described embodiment, the comparison can be performed by an external device.

The installation state determination system according to the present embodiment is configured such that whether there is an air layer between the probes and the living body is determined using the values of the thermal resistances of the living body at the time of the initial calibration when the probes are attached to measure a core temperature as described above. Thus, when the probes are not correctly attached, the user can be prompted to attach the probes again. As a result, it is possible to measure a core body temperature with little measurement error.

Although the embodiment of the installation state determination system and the installation state determination method of the present disclosure has been described above, the present disclosure is not limited to the described embodiment, and various types of modification that can be conceived by a person skilled in the art can be made within the scope of the invention described in the claims.

### REFERENCE SIGNS LIST

1 Installation state determination system

100, 200 Temperature measuring device

110, 120, 210, 220 Probe

130, 230 Thermal conduction member

400 Living body.

1-8. (canceled)

9. An installation state determination method comprising:
measuring a temperature and a heat flux of a surface of a living body using a sensor installed at a predetermined site of the living body;
calculating a thermal resistance value of the living body based on the temperature and the heat flux of the surface of the living body;
comparing the thermal resistance value of the living body with a reference thermal resistance value of the predetermined site of the living body; and
determining an installation state of the sensor at the predetermined site of the living body based on a result of comparing the thermal resistance value with the reference thermal resistance value.

10. The installation state determination method according to claim 9, wherein
when a difference between the thermal resistance value of the living body and the reference thermal resistance value exceeds a predetermined threshold, it is determined that there is an air layer between the sensor and the living body.

11. The installation state determination method according to claim 9, wherein
the thermal resistance value of the living body is calculated based on the temperature and the heat flux of the surface of the living body and a reference core body temperature of the living body at a start time of measuring the temperature and the heat flux of the surface of the living body.

12. The installation state determination method according to claim 9, wherein
the reference thermal resistance value of the predetermined site of the living body is determined in advance for a plurality of thicknesses of the living body.

13. An installation state determination system comprising:
a sensor installed at a predetermined site of a living body, the sensor being configured to measure a temperature and a heat flux of a surface of the living body; and
a computation device configured to:
calculate a thermal resistance value of the living body based on the temperature and the heat flux of the surface of the living body;
compare the thermal resistance value of the living body with a reference thermal resistance value of the predetermined site of the living body; and
determine an installation state of the sensor at the predetermined site of the living body based on a result of comparing the thermal resistance value of the living body with the reference thermal resistance value.

14. The installation state determination system according to claim 13, wherein when a difference between the thermal resistance value of the living body and the reference thermal resistance value exceeds a predetermined threshold, it is determined that there is an air layer between the sensor and the living body.

15. The installation state determination system according to claim 13, wherein
the thermal resistance value of the living body is calculated based on the temperature and the heat flux of the surface of the living body and a reference core body temperature of the living body at a start time of measuring the temperature and the heat flux of the surface of the living body.

16. The installation state determination system according to claim 13, wherein
the reference thermal resistance value of the predetermined site of the living body is determined in advance for a plurality of thicknesses of the living body.

17. An installation state determination method comprising:
measuring a temperature and a heat flux of a surface of a living body using a sensor installed at a predetermined site of the living body;
calculating a thermal resistance value of the living body based on the temperature and the heat flux of the surface of the living body;
comparing the thermal resistance value of the living body with a reference thermal resistance value of the predetermined site of the living body; and
determining whether there is air between the sensor and the living body at the predetermined site of the living body based on a result of comparing the thermal resistance value with the reference thermal resistance value.

18. The installation state determination method according to claim 17, wherein
the thermal resistance value of the living body is calculated based on the temperature and the heat flux of the surface of the living body and a reference core body temperature of the living body at a start time of measuring the temperature and the heat flux of the surface of the living body.

19. The installation state determination method according to claim 17, wherein
the reference thermal resistance value of the predetermined site of the living body is determined in advance for a plurality of thicknesses of the living body.
